(19) United States
(12) Patent Application Publication
(10) Pub. No.: US 2016/0029949 A1
(43) Pub. Date: Feb. 4, 2016

(54) APNEA AND HYPOVENTILATION ANALYZER

(71) Applicant: TECHNION RESEARCH & DEVELOPMENT FOUNDATION LTD., Technion City, Haifa (IL)

(72) Inventors: Amir LANDESBERG, Haifa (IL); Dan WAISMAN, Haifa (IL); Jimy PESIN, Haifa (IL); Lior LEV-TOV, Haifa (IL); Anna FINEGERSH KLEBANOV, Haifa (IL); Carmit LEVY, Sde Eliezer (IL)

(21) Appl. No.: 14/779,978

(22) PCT Filed: Mar. 25, 2014
(86) PCT No.: PCT/US14/31691

§ 371 Date: Sep. 25, 2015

Related U.S. Application Data
(60) Provisional application No. 61/804,767, filed on Mar. 25, 2013.

(51) Int. Cl. A61B 5/00; A61B 5/11

(52) U.S. Cl. CPC A61B 5/4818; A61B 5/1126; A61B 5/7246; A61B 5/7264; A61B 5/7278; A61B 5/7225; A61B 5/7282; A61B 5/6823; A61B 2560/0475; A61B 2562/0219

(57) ABSTRACT

A device is operative to quantify the severity of changes in ventilation strength and amplitude by monitoring the effort and efficiency of breathing activity, and classifying the source of apnea as either central or obstructive.

### FIELD OF THE INVENTION

The present invention is related generally to monitoring, detecting, quantifying and classification of apneic episodes, hypoventilation or increased respiratory effort and the like.

### BACKGROUND OF THE INVENTION

Apnea and hypoventilation cause hypoxemia and cyanosis, and arrhythmias if prolonged. It causes accumulation of irreversible damage, depending on the nature (suffocation, obstruction, central or mixed), frequency and severity of the episodes, and may lead to death. There is a need for monitoring apnea in infants admitted to neonatal intensive care units (ICUs), newborn care departments, intermediate care units, pediatric ICUs and pediatric departments. There is also a growing need for monitoring and classification of apnea episodes in sleep laboratories, home monitoring of babies at risk for hypoventilation, apneic episodes, suffocation or strangulation in bed and adults with sleep apnea.

There are several types of hypoventilation and apnea:

(i) Central apnea: Characterized by a sudden cessation or insufficient neural stimulation from the respiratory centers in the brain (medulla) to the respiratory muscles, leading to diminished or ceased breathing. Central apnea has multiple mechanisms: a depressed central ventilatory output, changes in thresholds for sleep or arousal, or immaturity of the brain's (medullary) respiratory control center.

(ii) Obstructive apnea: This apnea results from obstruction in the upper airways (due to collapse of soft tissue in the oropharynx, discoordination and relaxation of the buccal and pharyngeal muscles), backward movement of the tongue (due to inactivity of the genioglossus muscle), severely infectious disease, nasal occlusion and laryngospasm. Other causes are anatomical factors, such as enlarged tonsils or adenoids.

(iii) Mixed apnea: Obstruction and respiratory effort both initially stop in such an episode, followed first by a resumption of unsuccessful respiratory effort.

(iv) Accidental suffocation and strangulation in bed (ASSB). ASSB includes suffocation by soft bedding or pillow, rolling on top infant, entrapment of an infant between objects, asphyxiation, entrapment of the head between objects, or alike.

The main needs are:
Sudden infant death syndrome (SIDS) and accidental suffocation and strangulation in bed (ASSB). SIDS is defined as the sudden death of an infant under 1 year of age and unexplained even after comprehensive investigation (including autopsy). The incidence of SIDS is 0.543 deaths per 1,000 live births in the USA (2005), and is identified as one of the leading causes of infant death in the USA. ASSB deaths share many characteristics as sudden infant death syndrome (SIDS) but if has a separate classification. About 12.5 infant deaths per 100,000 live births are attributed to ASSB, in the USA. ASSB is a leading category of injury-related sudden unexpected deaths, these episodes are potentially preventable. These unexpected sudden infant deaths are also the leading cause of death in healthy infants after one month of age.
Apnea of prematurity: Defined as an unexplained episode breathing cessation longer than 20 seconds, or of shorter duration if symptomatic (decreased heart rate or desaturation). It generally refers to infants with a gestational age of less than 37 weeks. These neonates are hospitalized and require continuous monitoring. Apnea of prematurity may be central or obstructive, but most commonly mixed.
Apparent life-threatening event (ALTE): Defined as an episode that is frightening to the observer and characterized by some combination of apnea (central or obstructive), color change, marked change in muscle tone (usually marked limpness), choking, or gagging. Up to 0.9% of infants will have apnea that leads to admission to hospital. Thirty-second apnea occurs in 2.3% of healthy infants and in 13.1% of patients with a history of idiopathic ALTE.
Infants with medical indication for home monitoring: Defined as an infant with severe bronchopulmonary-dysplasia (BPD) and previous apnea episode(s), or that has a sibling that died from SIDS, or that had an episode of apparent life-threatening event (ALTE).
Home ventilatory support programs or home oxygen therapy.
Home monitoring: Mostly families using devices for home monitoring, since parents are in need of continuous medical reassurance, and fear the possibility of SIDS.
Obstructive sleep apnea in children: Airway obstruction is frequently produced by adenoidal or tonsilar hypertrophy. Diagnosis of this obstructive sleep apnea is important as an indication for surgical intervention.
Sleep apnea in adults: There is a growing awareness of sleep apnea in adults. During an episode, the PO₂ may fall as low as 20 mmHg, with saturation below 50%, causing severe hypoxia. Such an event is very stressful, increasing sympathetic activity. Consequently, many studies have shown that sleep-apnea increases blood pressure, and is associated with an increased prevalence of stroke and heart attacks. Patients with sleep apnea also exhibit severe arrhythmias (bradyarrhythmias during apneic episodes and tachyarrhythmias when breathing resumes). The condition also affects quality of life by causing headaches, daytime hypersomnolence, difficulties at work and automobile accidents. To prevent these harmful effects, it is recommended that a patient with severe sleep apnea due to obstruction will be treated with continuous positive airway pressure (CPAP), as this treatment can reduce the incidence of myocardial ischemia.

### SUMMARY OF THE INVENTION

The present invention seeks to provide a device that continuously monitors the patient and detects episodes of apnea, hypoventilation or increased respiratory effort and the like, and which also quantifies the frequency and duration of the apnea events, and also classify the various episodes, as is described more in detail herein below.

In accordance with an embodiment of the present invention, the device has (i) the ability to provide fast detection of changes in the tidal volume and in the respiratory effort—that is required for the immediate intervention. (ii) the ability to quantify the severity of changes in ventilation strength by monitoring the effort and efficiency of breathing activity, and (iii) the ability to classify the type hypopnea \ apnea as either central or obstructive and to differentiate it from other physiological increase in the effort, as emotional stress. The classification of apnea is based on measuring the respiratory effort and characterizing the changes in the shape and structure of the inspiratory phase.

The device has a wide variety of applications, such as but not limited to, in-hospital at-home patients, sleep-labs, and ambulatory usage; infants at risk of sudden infant death syndrome (SIDS), infants at risk for apnea (e.g., premature infants, following or during bronchiolitis, etc.); children and adults that suffer from obstructive sleep apnea or diseases that affect control of breathing (e.g., Ondine Curse, following cerebro-vascular accidents, etc.).

The device alerts about apnea or hypopnea episodes, quantifies their severity and classifies their nature (central, obstructive or mixed), in order to expedite the required intervention and to enable providing the correct treatment. For example, in the case of central or mixed apnea, the infant should receive methylxantines (caffeine, aminophylline), obstructive apneic episodes relate to other problems (such as severe hypotonia, tumoral lesions, gastroesphageal reflux, etc.) that require different treatments, and in case of ASSB—early detection will require simple repositioning of the head and the neck in most of the cases, while late detection will require resuscitation.

### BRIEF DESCRIPTION OF THE DRAWINGS

The present invention will be understood and appreciated more fully from the following detailed description, taken in conjunction with the drawings in which:

FIG. 1 is a simplified illustration of a device for monitoring and detecting apnea, constructed and operative in accordance with an embodiment of the present invention. The system may detect also the heart rate, as an adjuvant index for the severity of the situation.

FIG. 2 is a simplified illustration of a method for monitoring and detecting apnea, in accordance with an embodiment of the present invention. The displayed indices are only part of the monitored indices, as described below.

FIG. 3: Filtered raw data from a typical demonstrative experiment performed (in rats) during an obstructive apnea episode that lasted 23 seconds. Complete airway obstruction was imposed at sec 155, and released at sec 178. Subplots from top to bottom: Mean Arterial Blood Pressure (MABP), Pulse Oximetry (SpO₂), End tidal CO₂ (EtCO₂), Esophageal Pressure (EP) and Heart Rate (HR). The tidal displacement index (TDI) signals from the Right (R), Left (L) and Abdominal (Ab) sensors are displayed. Baseline recording of 30 sec preceded a further significant increase in the respiratory effort against the imposed obstruction. The EP and the tidal displacement index (TDI) signals showed a good correlation during the episode. Following the release of the obstruction the recovery of the vital signs and the tidal motion signal patterns returned to baseline values.

FIG. 4: Raw filtered data from an event mimicking Hypopnea/central apnea (CA) induced by IV injection of succinylcholine (left arrow). The decrease in the respiratory efforts induced hypopnea (mid arrow) that approached a 50% decrease in respiratory effort compared to baseline (as demonstrated by the EP) and progressed to a total cessation of breathing attempts (right arrow). Subplots from top to bottom: Mean Arterial Blood Pressure (MABP), Pulse Oximetry (SpO₂), End tidal CO₂ (EtCO₂), Esophageal Pressure (EP), Heart Rate (HR), and the motion signals (TDIs) from the Right (R), Left (L) and Abdominal (Ab) sensors.

FIGS. 5A-5B: Tidal Amplitude (TA)—(FIG. 5a) and Esophageal Pressure (EP)—(FIG. 5b), Mean±SD in obstructive apnea (rectangles) and central hypopnea-apnea (rhombus) episodes. Obstruction or progressive paralysis was induced at time “0”. While −10 to −1 are the baseline measurements. Points 10 to 19 represent absent respiratory movements during Hypopnea/CA. Values are expressed as relative changes from baseline of a normalized Tidal Displacement index (TA in the Y axis). Detection and quantification of the respiratory effort and amplitude are shown for both, Obstructive (increase in the TDI) or Hypopnea and central Apnea (decrease in the TA). Need to be noted from one side, the significant difference between baseline, obstructive apnea (OA), hypopnea of central Apnea (CA), and the similar responses recorded using the TA (a) and the ER. The different behavior of the TAduring AO and Hypopnea/CA, enables an easy and clear classification of both types of episodes and their severity.

FIG. 6: Mean±SD of the Breath Time Length (BTL). Complete airway obstruction and hypopnea were induced at point 0. Both, obstructive and Hypopnea/Central Apnea induced a marked increase in BTL.

FIG. 7: Amplitude Time Integral (ATI−Mean±STD) obtained from the right and left sensors during airway obstruction and hypopnea/CA. The ATI showed a significant progressive increase as the result of airway obstruction (with mild difference between the right and left sides of the chest that was not statistical significance). The ATI showed also overt progressive decrease in respiratory efforts during Hypopnea/Central Apnea. The quantification of the changes in the work of breathing is feasible utilizing this index.

FIG. 8: Raw filtered data from an event of Fatal Obstruction. Obstructive apnea that lasted 32 seconds (sec 228 to 260) leads to the development of fatal central apnea episode (sec 260 to 278). The obstructive apnea induced severe hypoxemia, which caused brain hypoxia and malfunction of the central respiratory center. Consequently, it evolved into irreversible central apnea with no respiratory effort. There was a need for resuscitation to keep the animal alive. The respiratory movements seen from sec 278 were the result of manual ventilation provided with a self-inflating bag, during the resuscitation.

FIG. 9. The vital signs during one experiment in rabbit containing all eight events (from left to right): three hypoxic events that mimik severe stress (decrease in the oxygen partial fraction in the inspirate air from the normal fraction of 21% to 16%, 14%, 12% of O₂), two full obstructions, and two partial obstructions (that were associated with 50% and 25% increase in the esophageal pressure), and one central apnea event. BP-mean blood pressure, SpO2—Pulse Oximetry, Eso. Pr.—Esophageal pressure, an invasive gold standard for the respiratory effort, RR— Respiratory rate.

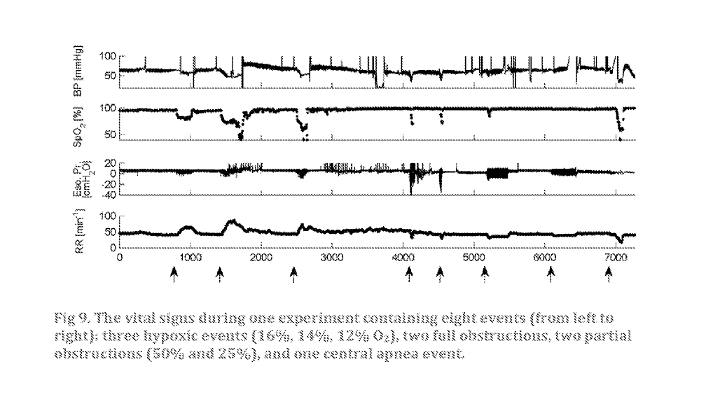

FIG. 10. Five seconds of the overved raw data in our sensors during (A) baseline, (B) mild (25%) Obstruction, (C) and moderate stress induced by hypoxia (decreasing the O₂ partial fraction to 16%). Note the overt changes in the shape of the signals, and especiallty the difference between mild obstraction and the baseline or stress.

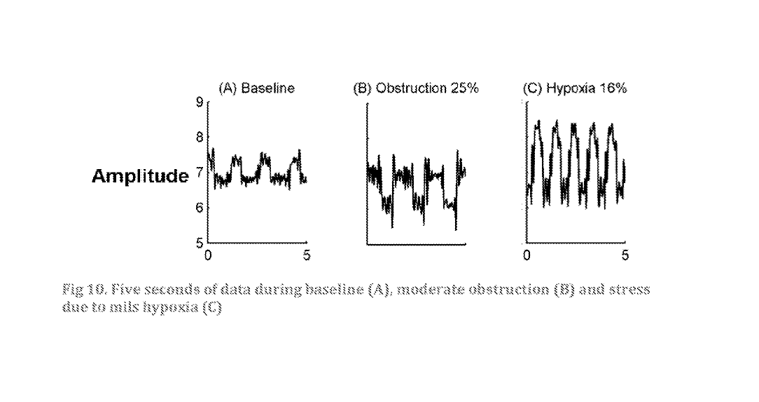

Each parameter demonstrates a similar response for all the different events.

FIG. 11. The Kurtosis and the variance parameters measured from the sensors. The time scale and the events are identical to FIG. 9. The Variance describes the changes in the amplitude of the respiratory effort. It increases during hypoxia and onstraction (all the first 7 events) and decreases during central apnea. The Kurtosis quantiify the changes in the shape of the signals and show minimal changes during hypoxia (the first three events) however increase during obstructive apneas. Thus the kurtosis, from a single sensor, can diffrentiate between a simple increase in the effort that is not associate with obstraction or changes in the respiratory system mechnains and an increase in the effort that is due to obstraction or increase in the resistance to flow within the airways.

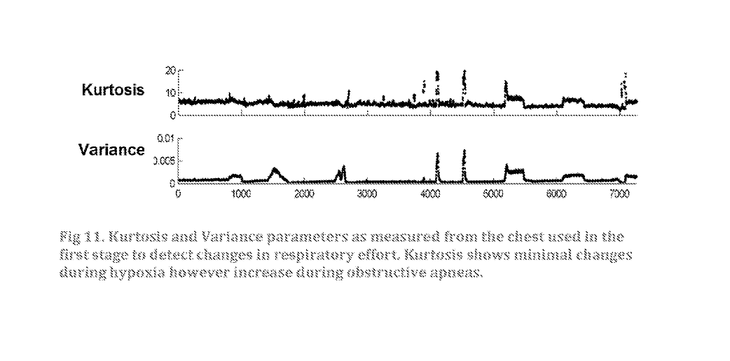

### DETAILED DESCRIPTION OF EMBODIMENTS

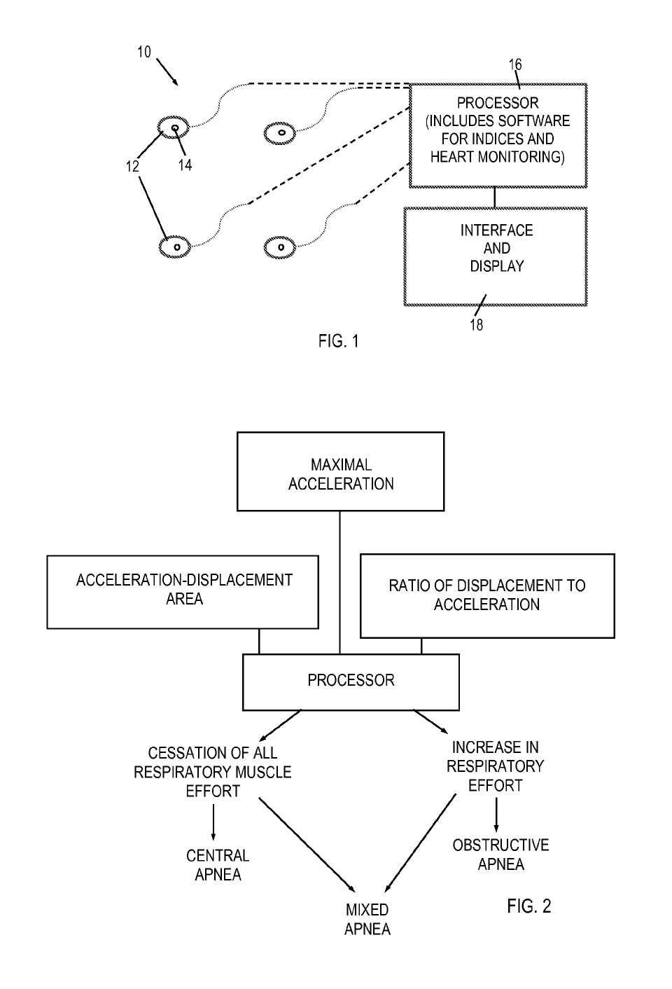

Reference is now made to FIG. 1, which illustrates a device 10 for monitoring and detecting apnea (also referred to as apnea analyzer), constructed and operative in accordance with a non-limiting embodiment of the present invention.

The device 10 continuously monitors chest wall dynamics, and in addition measures the respiratory effort and the apparent efficacy of respiration. It can detect and analyze the different types of apnea from a single sensor 12 on the chest. In the present invention, sensor 12 is an accelerometer. Other sensors as gyro-meters or gyroscope, that sense and quantify the motion of a single point in the space, can be used (all three types of sensors are referred to as a local acceleration sensor). Other accelerometers or alike 12 may be used, such as on the abdomen. The sensors may be embodied in a patch, referred to as a patch and sensor unit.

The system includes in a non-limiting embodiment:
the hardware for data amplification and filtration.
the required data acquisition system and data storage for continuous data analysis and comparison with the past.
The system includes central processing unit (processor 16) (that may include additional channels for ECG acquisition with appropriate gain (1000) and filtering (0.05 to 120 Hz)).
Software for analyzing the respiratory effort indices.
May include software for heart rate monitoring (not necessarily from the ECG)
User-friendly interface and display 18 of the new indices for detection, classification (central or apnea) and quantification of the severity (frequency, duration, asynchrony, efforts) of the apnea episodes.

The system may also include contact electrode 14 for ECG (EMG), which may be in each patch and sensor unit.

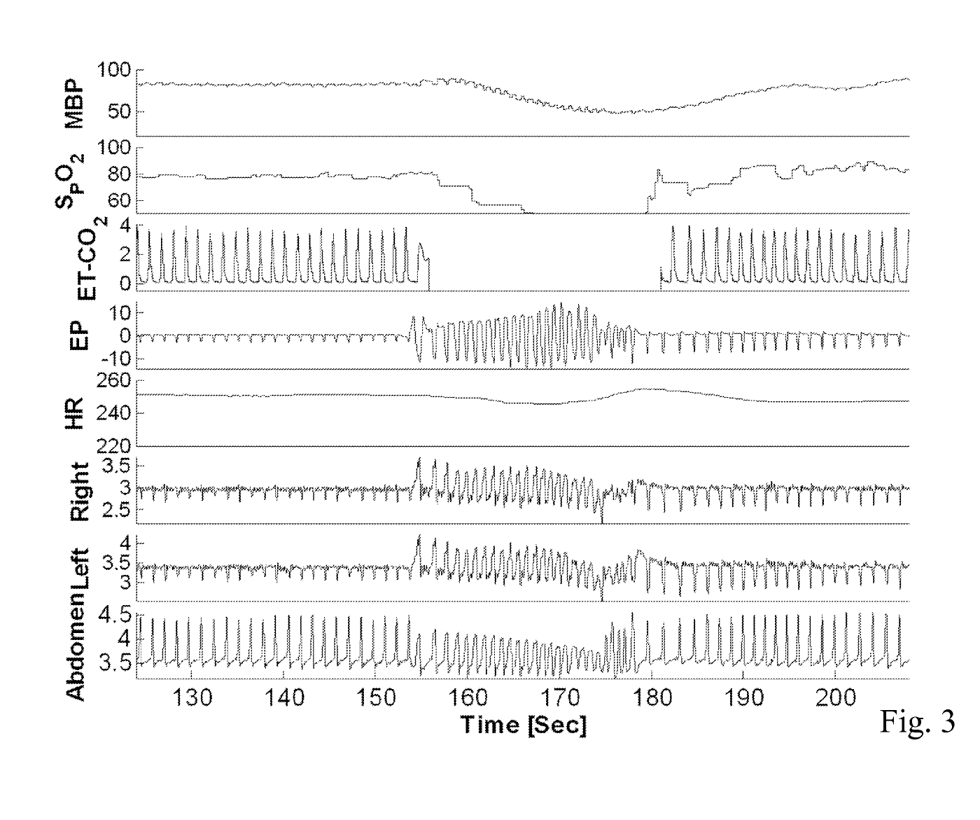

All the sensors are connected to processor 16. Processor 16 processes the sensed local accelerations and\displacement and\or motion of each sensor and quantify the severity of changes in ventilation strength by monitoring the effort and efficiency of breathing activity, and thereby classify the source of apnea as either central or obstructive, as will be described now with reference to FIG. 2.

The following is noted:

(i) According to Newton's second law, force is proportional to mass and acceleration, therefore measurement of the acceleration provides crucial information about the respiratory effort (force), especially since the mass of the chest remains constant.

(ii) All available apnea monitors of the prior art are based on measuring the chest displacement (in most devices) or the upper airway flow velocity. It is technically impractical to derive the acceleration and forces from these measurements due to relatively large noise. Thus, in contrast to the prior art, measuring acceleration is more informative. Moreover, it is possible to integrate the acceleration and calculate the flow and displacement

(iii) In obstructive apnea, the main factor that determines the severity of hypoxia, the degree of activation of the sympathetic system and the severity of stress is the work required to overcome an obstruction. Thus, the best way to quantify patient complaints (dyspnea) is by quantifying the work required for adequate respiration.

(iv) Classification of the source of apnea, central or obstructive, is based on measurement of the breathing muscle activity. In central apnea there is cessation of all respiratory muscle effort, while in obstructive apnea there is even an increase in the efforts of the respiratory muscles.

(v) The measurements are sensitive to possible changes in the respiratory muscle work. Such changes include the energy dissipated as heat due to flow resistance through the obstruction, energy losses due to turbulence and chest wall viscous properties, and the energy stored in the elastic properties of the lung and chest wall.

(vi) The phase differences between the dynamics of the chest sensor and the abdominal sensor (i.e., between the three moments in the different sensors) provide additional information used to classify the various types of apnea.

The mechanics of the respiration is determined mainly by the respiratory muscle effort, the lung and chest wall compliances, the resistance to flow in the airway system, and other secondary viscous and non-linear properties of the lung, chest wall, muscles and the flow through the airways. Knowing the instantaneous acceleration, flow and volume utilizing our sensors can be used to approximate the required respiratory effort.

Thus, the device enables detecting not only the respiratory rate and changes in tidal volume but also changes in the effort, efficacy and synchrony of the ventilation, to assess the effect of obstruction, and to quantify the severity of the obstruction. This is the first device that can quantify the effort of the patient and detect a reduced efficacy of ventilation. The effort is assessed by several novel indices as the maximal acceleration, velocity-displacement area (in analogue to the flow-volume plots that are used in plethysmography), acceleration-displacement plots (in analogue to pressure-volume plots that are used to assess the respiratory work, the ratio of displacement to acceleration (D2A), the tidal displacement amplitude, the duration of the breath signal (breath time length) the amplitude time integral (in analogue to the force-time integral used for assessing skeletal muscle energy consumption), assessment of the energy and entropy in the recorded signals, and other.

All these indices can be derived from a single sensor on the chest.

An additional important factor is the synchrony between the dynamics of the chest and the diaphragm, which is normally tight. During obstruction, since the chest volume is practically constant the motion of the chest and the diaphragm are slightly out of phase or in complete opposite phase.

Continuous monitoring of the respiratory effort, efficacy and synchrony of the respiratory muscles' function provide significantly more precise (and currently unavailable) indices characterizing the work of breathing. Precise assessment of the effort of respiration enables to differentiate between central and obstructive apnea and hypopnea.

Current prior art technologies for apnea monitoring: Diagnosis of apnea is currently made by visual observation or by use of multichannel monitoring of cardiorespiratory functions. There are four main groups of technologies: 1. Sensor over the chest and abdomen to monitor changes in chest volume, 2. sensors at the nostrils to monitor the air-flow or fluctuation in CO₂, 3. monitoring the end results of respiratory function by oxymetry and hemodynamic indices, and 4. monitoring the respiratory motion by remote sensors placed under the patient's mattress or in a vicinity to the patient.

### 1. Monitoring the Changes in Chest Volume:

Transthoracic electrical impedance monitors: this is the prevailing technology in most devices today. These monitors measure changes in electrical conductivity between two or more electrodes attached to the chest.
Respiratory inductive plethysmography (RIP): Coiled wires sewn into an elastic belt are placed around the chest and abdomen. A current applied to the coils generates a magnetic field. Changes in the cross-section area within these loops induce electromotive force (EMF, Faraday's Law). These systems are used in sleep and research labs, and are not in routine use in hospitals or as ambulatory monitors.
Elastomer (strain gauge) plethysmography: Elastic bands with stain gauges are placed around the chest and abdomen. These systems are relatively cheap and are simple to use.

All these monitors are efficacious in identifying central apneas. The identification of obstructive apnea in these systems was suggested by monitoring the development of asynchronous motion between the chest and the abdomen, but these systems suffer from severe limitations in the ability to identify obstructive or mixed apnea for the following reasons:
Obstructive and mixed apneas may not be detected in time by these monitors since the patient tries to overcome the resistance to flow and even increase the effort of respiration and chest wall motion. Hence, diminished breath movements in the case of obstruction may be a late sign of fatigue and already severe desaturation.
The analysis and quantification of the degree of asynchrony is qualitative and not well established. Therefore, the ability to detect obstructive apnea based on this index is not precise.
In addition the plethysmographic systems are cumbersome. Moreover, any changes in the belt position, patient position or movements may produce severe problems: If the belts are too tight or too loose they will not detect the respiratory movements and even may limit chest expansion in weakened patients, such as prematurely newborn infants.

### 2. Monitoring the Nasal Air Flow:

The flow is monitored by nasal temperature, pressure transducers or CO₂ monitors.

These systems have several disadvantages:
Cannot differentiate between various types of apnea (in all types there is no air-flow in the upper airways).
Delay in detecting moderate partial obstructions, since the patient maintains flow by increased effort.
Do not provide quantitative or semi-quantitative data on lung function.
Are impractical for routine monitoring of infants (require attachment of devices to the face or airway)
Are impractical when the patient is breathing through the mouth (especially in adults)
The end tidal CO₂ may add dead space.

### 3. Monitoring the End Results—Oxymetry and Hemodynamic Indices:

The main disadvantages of these methods are:
a high false positive or false alarm rate.
Severe delay in diagnosis. Bradycardia and desaturation appear very late in events of apnea.
Cannot differentiate between central, obstructive and mixed apneas.

### 4. Monitoring Respiratory Movements by Remote Sensors.

These monitors are based on movement detection, with sensors located under patient's mattress or by using optical means for monitoring breath motion. These systems suffer from some limitations, rendering them unaccepted as accurate methods for apnea monitoring and without FDA approval.
There is a very high rate of false positive alarms when the infant is not moving in the range of the sensor or has shallow breathing. This has sent many infants to the emergency department because parents started resuscitation without carefully checking the baby.
It is associated with a detection delay, since during obstructive apneic episodes there is movement and these monitors will not detect obstructive apnea until the infant faints and is deeply hypoxemic.

Therefore, the above technologies cannot be used as a standalone system for detection of apnea. In contrast, some systems use simultaneous measurement of the following physiological indices: (i) volume (impedance/plethysmography), (ii) nasal air flow and, (iii) Heart rate (ECG). These combined measurements can be used to differentiate between central and obstructive apnea based on:
The synchrony of chest and abdominal movements.
The correlation between chest movement and nasal airflow.

Asynchrony between the chest and abdomen, and chest movement in the absence of nasal airflow are pathognomonic of obstructive apnea. Measurement of the heart rate is used as adjuvant index for the severity of the apnea.

However, these systems are cumbersome, imprecise, suffer from prolonged delay in detection of obstructive apnea and poor quantification of obstruction severity.

Technological Uniqueness:

The apnea analyzer of the present invention overcomes these limitations and provides significant advantages. The suggested system directly monitors the dynamics and uniformity of lung ventilation. The current technologies suffer from two main disadvantages: prolonged delay in detection of obstructive apnea and unclear classification of the apneic episode. The present innovation overcomes these two limitations and provides crucial information (increase or decrease in the effort, asymmetric ventilation, changes in volume, type of apnea) that improves the diagnosis.

The technology is unique and enables earlier detection of deterioration for the following reasons:

1. Directly measures and monitors the chest and lungs dynamics, and sensitive measurement of the respiratory effort.

2. Takes measurements of the entire frequency range from the low frequency of ventilation to the high frequency of breathing sounds.

3. Enables monitoring the ventilation volumes and flow velocities, and assesses the lung mechanical transfer function. Thus, it can determine whether there is normal airway resistence as in central apnea of increase in the airway resistence and in obstructive apnea.

4. Assesses the synchrony of the chest and abdomen movement. Central apnea is anticipated to preserve the synchrony between the two and obstructive apnea is associated with some asynchrony and sometime even with an image mirror between the two.

5. Adjusts to patient's baseline conditions. After the attending staff approves the baseline status, the device registers it and alerts of any change relative to that baseline. Its function is independent of the patient's initial condition.

6. It is unique in its physical principles, recorded signals and the data analysis algorithms.

Functional Uniqueness:

We have shown that early detection of deterioration in the ventilation is feasible in neonates (10 babies). We have tested the suggested system in pre-clinical studies. A simpler system that monitors only the amplitude of the tidal chest wall displacement was tested in neonates in the neonatal intensive care unit. The patients were simultaneously monitored with cardiorespiratory monitors, pulse oximeters, transcutaneous PO₂/PCO₂. Our technology detected the development of complication on average 22.4±18.7 minutes before the blood oxygen dropped below 90% and the patients became symptomatic. Moreover, it provided crucial information that could assist in the diagnosis of the underlying problem and also enabled the immediate assessment of the efficacy of the therapeutic interventions performed.

1. It was possible to detect the deterioration 22.4±18 7 minutes on average before a critical life threatening hypoxemic episode (desaturation) appeared and also before the newborn became symptomatic. This study established the feasibility to detect changes in the respiratory effort.

2. Provides additional, crucial information that facilitates correct diagnosis, leading to faster correct responses of the attending staff. The improvement in diagnosis was established in our clinical study.

3. Enables fast assessment of treatment by providing accurate assessments of the efficacy of mechanical interventions. It provides fast feedback on the effects of interventions on lung ventilation mechanics. The positive predictive value of the observed changes in mechanical indices was 94.7% in our clinical study.

4. Easy to use and understand. Although new indices are measured, it relates back to basic physiological indices.

5. Inexpensive (cost-effective).

The Apnea Analyzer overcomes the limitations of current apnea monitors by the following:

Technical Advantages:

1. The unique measurement of chest acceleration enables assessment of the equivalent force imposed in breathing. The calculated displacement and assessed forces enable to derive the respiratory effort work and the efficacy of the respiratory muscle work.

2. Direct assessment of the effort of ventilation is an essential parameter that cannot be derived from other available technologies that only measure the volume, flow, or breathing sounds.

3. Diagnosis can be assessed from a single sensor, since each sensor assesses the three moments of the chest dynamics. The information from a single sensor can be used to detect apneic episode and to differentiate between the various types of apnea.

4. Monitors the synchrony between the chest and abdomen in all three moments can be further improved by utilizing two or more sensors on the chest and upper abdomen.

Functional Advantages:

1. Earlier detection, as established in our preliminary study in ventilated patients. We immediately detected decreases in spontaneous breathing in case of central apnea. Earlier detection is easy in the case of central apnea, but is especially needed in obstructive apnea, where other methods fail.

2. The described innovation can also detect cessation of spontaneous ventilation when the patient is mechanically ventilated and even during high-frequency oscillatory ventilation (HFOV). This ability opens an avenue towards optimizing the mechanical ventilation.

3. Detection and classification of all types of apnea based on monitoring the effort of the breathing muscles, the apparent efficacy of respiration (the ratio of displacement to required effort), and the synchrony of respiratory movement.

4. Monitors directly the dynamics of lung ventilation. Any changes in the mechanics of lung ventilation, such as increases in flow resistance (obstructive apnea) affect the chest inflation and deflation dynamics.

5. It assesses the synchrony of ventilation between the chest and abdomen, as do the other devices, but with miniature patches and with higher sensitivity (since it measures the acceleration, a faster and more sensitive signal to phase changes.

6. Precise assessment of the severity of apnea, not only by measuring the duration, but also by registering the changes in effort required to maintain the adequate ventilation in obstructive apnea or hypoventilation. It also enables detection of severe hypoventilation, and not just the existence severe apnea.

The system of the invention has the following advantages, among others:
Noninvasive and simple to use technology.
Continuous monitoring of lung ventilation.
Miniature and convenient sensors (unlike the belts, set of microphones, nasal sensor or Plethysmographic studies)
Does not require the cooperation of the patient.
Monitor the synchrony of ventilation (especially in neonates and infants), a sensitive index for development of obstructive apnea.
Monitor the changes relative to defined baseline condition, and therefore are sensitive to changes.
Can be used with any patient initial condition, and for any patient.
Provides earlier detection of deterioration in ventilated patient (established in preclinical and clinical studies).
Improves the diagnosis of the underlying complication when desaturation developed (established in clinical studies).
Facilitates the assessment of the effects of therapeutic interventions on ventilation mechanics.
Enables detection of apnea episodes and quantification the severity of the episodes, as shown in the pre-clinical study described below.
Enables classification of apnea episodes to central or obstructive (or mixed) apnea, as shown in the pre-clinical study described below.
Monitors and evaluates the efforts, synchrony and efficacy of the respiration.
Provides indices that correlate with the severity of the diseases (unlike breathing sounds) and therefore very reliable.

### Pre-Clinical Feasibility Study

### Experiment 1

The inventors performed experiments to verify the efficacy of embodiments of the invention, as is now described.

The objective of the study was to explore the utility of continuous monitoring of the chest wall dynamics with miniature motion sensors for real time detection and classification of central and obstructive apneic and hypopneic episodes induced to spontaneously breathing rats.

### Methods

Male Sprague-Dawley rats (n=9) underwent episodes of obstructive and central apnea, following the approval of the institutional Animal Care and Ethics Committee. The rats were anesthetized using intra-peritoneal injection of ketamine, xylazine and acepromazine (ACP). A venous line was placed in the tail vein, for further drug injection. Rectal temperature was monitored and maintained at 36.0-37.5° C. using a heating pad. A femoral arterial line was placed for blood pressure monitoring and gas sampling (Roche OPTI CCA, Mannheim, Germany). A tracheostomy tube (PE-240 polyethylene) was placed and connected to a side stream end-tidal CO₂ (EtCO₂) monitor (Datex MultiCup, Helsinki, Finland).

Chest wall and abdominal displacement were continuously monitored using the described three miniature motion sensors that were fixed on the right and left sides of the chest (midclavicular line at the forth intercostal space) and epigastrium. Esophageal pressure (EP) was measured using a fluid-filled catheter (PE-50 polyethylene tubing) placed at the mid-esophagus and connected to a pressure sensor (Millar Instruments Inc., Houston, Tex., USA). Vital signs (ECG, BP, EtCO₂ and SpO₂) were displayed continuously and acquired by an anesthesia\ICU monitor (Datex Ohmeda Inc, Type CU8, Wisconsin, USA).

Experimental Protocol:

Fifteen minutes of stabilization followed animal preparation. Few episodes of obstructive apnea (PA) and Central Apnea (CA) were performed in each rat. Each event was preceded by 30 sec of baseline recording, and after each intervention the recording continued until a complete recovery to baseline values occurred according to all the vital signs.

Obstructive apnea was achieved by complete airway obstruction, with an occluded endotracheal tube connector. Each episode of obstructive apnea lasted no more than 30 seconds or until an overt decrease in HR, SpO₂, or BP occurred during the acute episode.

Central Hypopnea and Central Apnea were achieved by intravenous infusion of 0.1 mg/Kg succinylcholine, if apnea did not occur after the initial infusion, the same dose was repeated. The injection of succinylcholine induced progressive muscular paralysis mimicking central driven hypopnea, before it progress to complete apnea.

Overt hypopnea was defined as a decrease of more than 50% in the respiratory effort measured by the EP, in respect to baseline. Central Apnea was defined as the absence of respiratory efforts for at least 10 seconds.

The following three indices (out of the suggested indices) were used to differentiate between the two types of apnea

Tidal Amplitue Index (TA) (Also Referred to as Tidal Displacement Index (TDi)):

The TA represents the amplitude of the tidal local displacement during the breath cycle, at the site of measurement (right or left side of the chest and epigastric area).

Breath Time Length (BTL):

is the total duration of the inspiratory and the rapid expiratory phases in each breath cycle signal.

Amplitude Time Integral (ATI):

The ATI is the integral of the instantaneous tidal displacement over the time, along the entire respiratory cycle.

Data management and analysis was performed using MATLAB® (R2009a, MathWorks Massachusetts, U.S.A.) and Excel (Microsoft Office 2007). Results are presented as Mean±STD.

Statistical Analysis:

Differences within groups were assessed using the paired Student's T-test. A P-value smaller than 0.05 was considered as significant.

### Results

Nine rats (313±46 g) were used in this study. Altogether, 43 OA events and 14 Hypopnea/CA events were performed. However, all the mean results from each rat were considered as a single experiment.

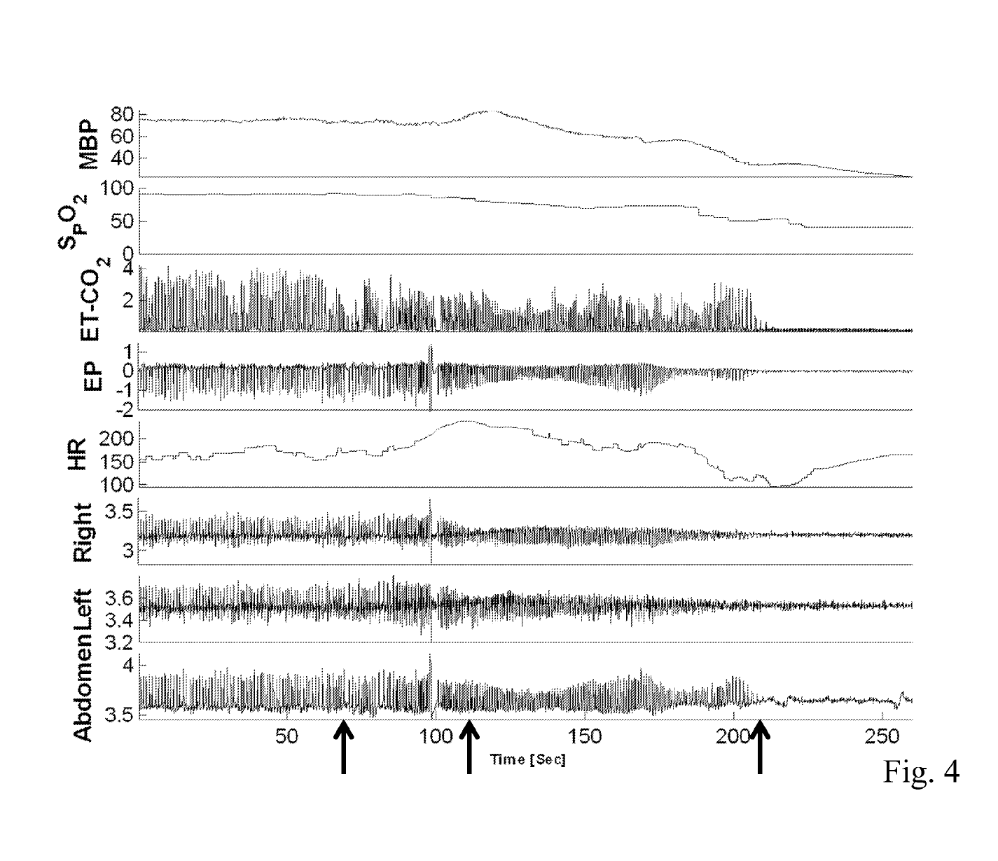

FIG. 3 presents the raw data from one OA experiment. The five signals present the regular measurement available in the clinic: Mean blood pressure (MBP), pulse oximetry (SpO₂), end tidal CO₂ (ET-OO₂), Esophageal Pressure (EP), and the Heart Rate (HR). It is important to note that the EP is impractical, and rarely used in the clinics. The last three traces present the output of the suggested motion sensors attached to the right (Right) and left (Left) side of the chest, and to the upper abdomen (Abdomen). The OA was imposed at 153 sec from the initiation and was released at 177 seconds. The motion sensor signals corresponded well to the EP deflections during baseline, obstruction and recovery periods. During the occlusion there was no EtCO₂ signal detection as the result of the imposed obstruction. The EtCO₂ showed an expected short delay imposed by the time requires shifting the sampled air from collecting tube to the sensor within the air gas monitor. Typical changes during OA included a significant, progressive increase in amplitude, and widening of the breath cycle, with an increase in respiratory rate.

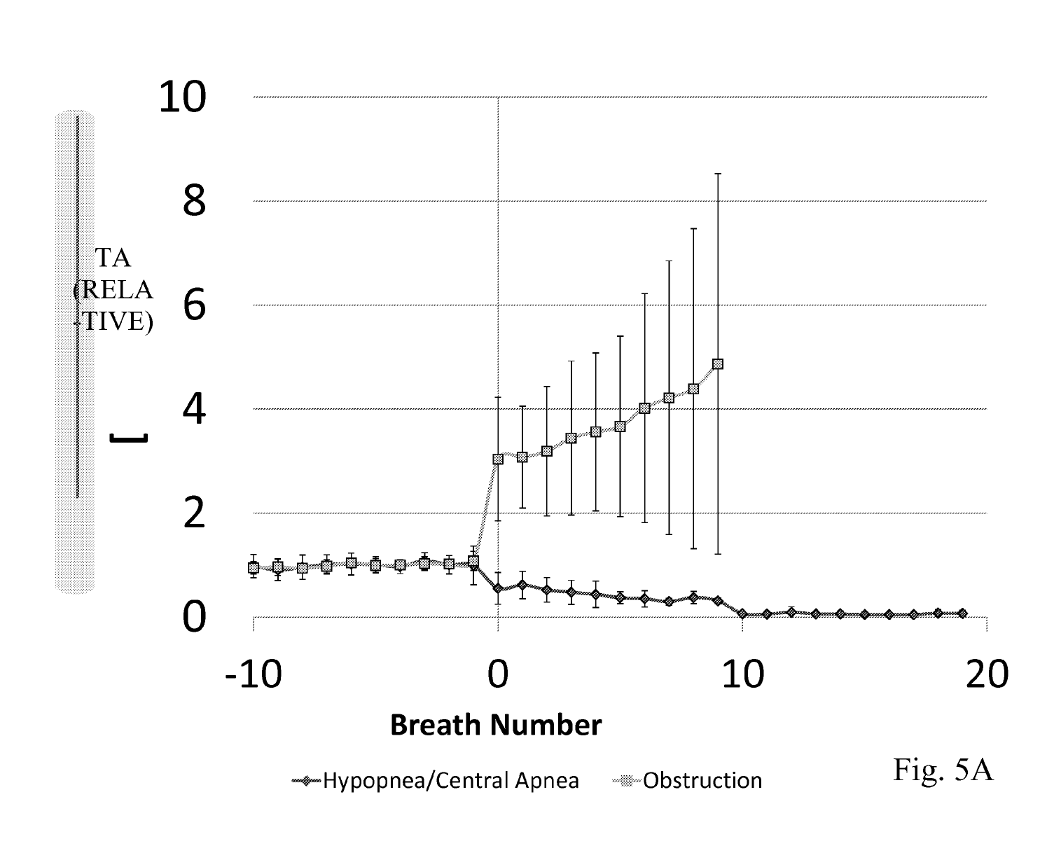

FIG. 4 presents the raw data from Hypopnea/CA episode. IV succinylcholine injection was started at t=71 sec (left arrow) and it induced a progressive decrease in the respiratory effort resulting in hypopnea (middle arrow) followed by apnea with no breath signals at t=208 sec (right arrow).

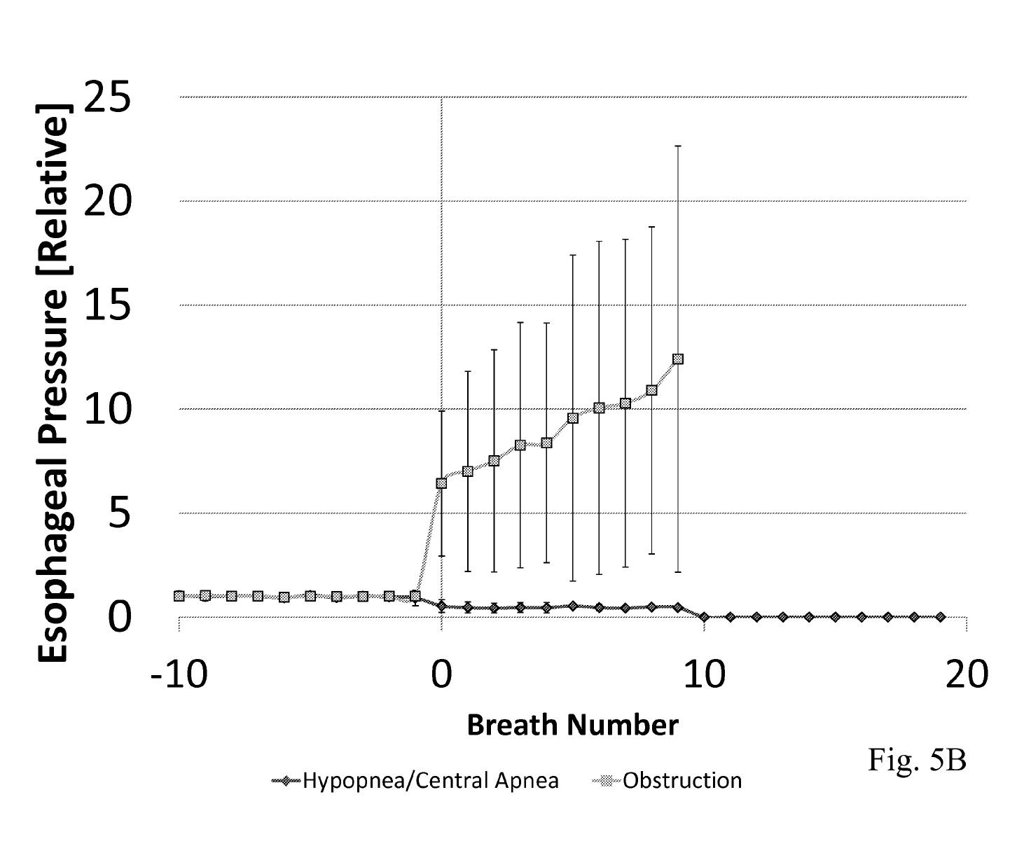

TA: FIG. 5a compiles the Mean±SD of the TA measurements from the obstructive and central apnea experiments. OA: An overt 3.75±1.87 fold increase (P<0.0001) in TA relative to the baseline level was obtained during the OA. FIG. 5b display the EP amplitude, which significantly increased during the obstruction episodes by 9.08±6.6 fold (P<0.005). The changes induced in the EP by the obstruction were well in concordance to those observed in FIG. 5a for the TA in both, OA and Hypopnea/CA.

The response of the TA was clearly different for the OA and the CA episodes as shown in FIG. 5a, allowing for a clear discrimination of the type of event recorded when compared to baseline. Similar changes were obtained from the right as well as from the left chest sensors in all cases (data not shown).

Breath Time Length (BTL):

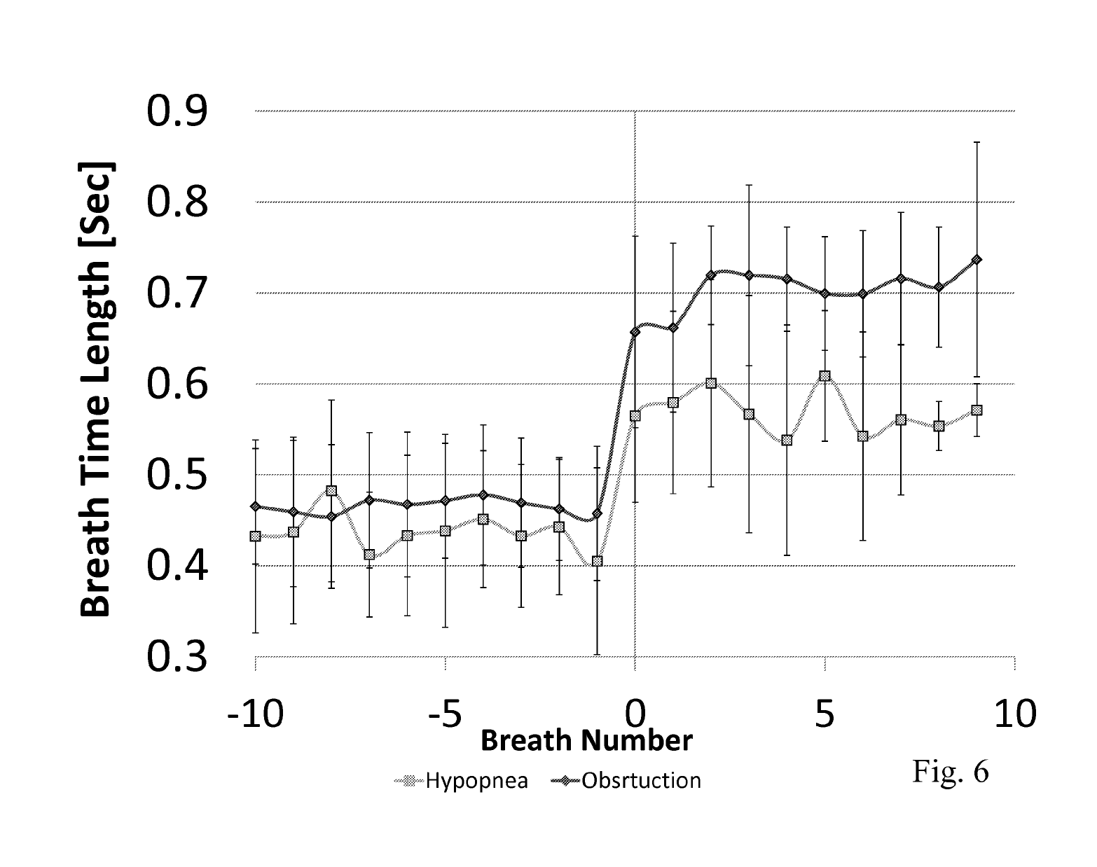

OA yielded a significant widening of the breath signals. At baseline the width of the breath signal was 0.47±0.065 sec, and it increased by 152±12.8% (P<0.0001) (FIG. 6). Interestingly, BTL increased also during the hypopneic period that preceded CA by 130.2±5.22% compared to baseline. This increase did not reach statistical difference when compared to the increase induced by airway obstruction.

Amplitude Time Integral (ATI)

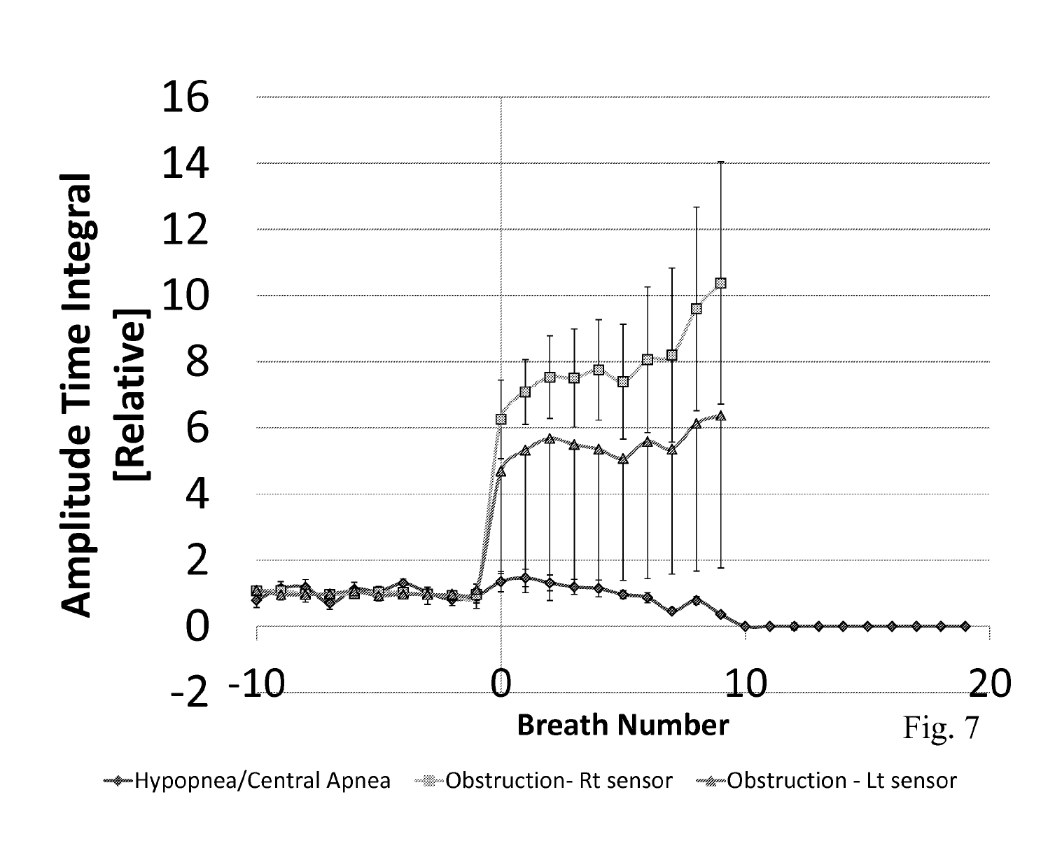

A significant change between baseline and airway obstruction was observed, with an increase of 5.5±4.06 times (P<0.005) for the left and 7.98±7.86 for the right side (P=0.014). FIG. 7 depicts the changes during the obstruction and hypopnea episodes in respect to baseline. An Interesting finding was the different behavior of the right and the left sides of the chest, while the ATI measured from the right side was 2.62±3.84 times greater than the ATI measured on the left side. The development of central apnea was slower (it takes time to succinylcholine to reach the desired effect) and takes about 10 breath cycles. As CA evolved the decrease in the ATI became evident.

Table 1 summarizes the utility and stability of the observed changes in the indices used in this research and the different responses during the two types of apnea.

Near-Fatal Obstruction Episodes:

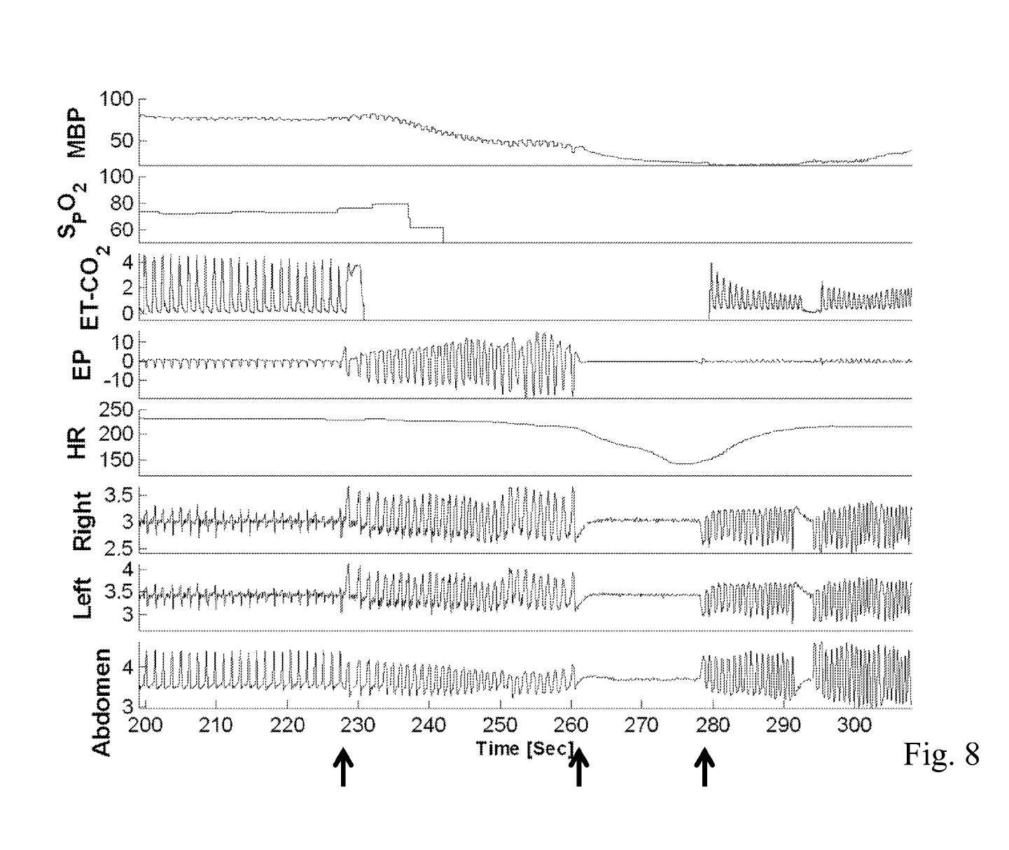

Six of the 43 events of airway obstruction (OA), evolved into a complete cessation of the breathing efforts as in CA. These events were the result of a severe decrease in BP, bradycardia and decrease in the SpO₂. In FIG. 8, a typical case is depicted. In all cases manual ventilation with a self-inflating bag was provided in order to recover the rat from the event. Although the obstruction was rapidly removed, arousal and subsequent spontaneous recovery did not occur.

### Discussion

The study demonstrated the feasibility and utility of a novel non-invasive monitoring system for real time detection and classification of apneic episodes. The system, based on miniature motion sensors, provided continuous information on chest wall mechanical changes and showed high accuracy in the detection and quantification of hypopnea, and early recognition of airway obstruction before they progress to respiratory failure.

The detection, characterization, and classification of the apneic episodes were performed using three parameters: Tidal Displacement index (TDi), Breath Time Length (BTL), and Amplitude Time Integral (ATI). Obstructive apnea induced 3.75±1.87 fold parallel gradual increase in TDi (P<0.0001), 1.52±0.02 times increase in breath-time length (P<0.0001), and 7.98±7.86 fold growth in the amplitude-time integral (P<0.0001). During hypopnea/CA episodes each sensor revealed overt gradual and quantifiable decrease in TDi progressing to a complete cessation of breathing attempts. The measured changes in these indices paralleled the changes in the EP.

With these parameters we were able to quantify also the severity of the decrease in respiratory effort during hypopnea that preceded central apnea.

A good correlation was observed between the EP and the TDi, showing that the TDi could be used as a simple and non-invasive surrogate of the EP for quantification of the respiratory effort. This correlation was observed during the obstructive apnea events as well as during Hypopnea/CA. Different invasive and non-invasive alternatives to EP for the assessment of the respiratory effort during OA were suggested, such as Pulse Transit Time, Diaphragm Electromyogram, Nasal airflow and Respiratory Inductance Plethysmography (RIP). However, none of these methods were accurate or simple enough for routine clinical use, especially in small pediatric patients.

Monitoring the chest wall dynamic changes using the mechanical indices presented here allowed a precise detection of the initiation and progression of all apnea events. During airway obstruction as well as for Hypopnea/CA, the beginning of the events could be clearly identified relaying only in this non-invasive set of measurements: the TDi, BTL and ATI that showed typical changes for each one of them.

Airway obstruction produced also a significant increase in the duration of each breath as a consequence of the increased effort recruited in order to overcome the obstruction. BTL alone, showed a significant increase, mainly by prolongation in the inspiratory time, as reported by Mooney et. Al. (Mooney A M, Abounasr K K, Rapoport D M, Ayappa I. Relative prolongation of inspiratory time predicts high versus low resistance categorization of hypopneas. J Clin Sleep Med 2012; 8(2):177-85). Mooney and coworkers used the measured slope of the nasal airflow during inspiration in Obstructive and Central Apnea, as well as during the high or low resistance hypopneic periods. They showed that an increase in inspiratory time provided the best correlation with OA, with a sensitivity and specificity of 84% and 74% respectively for the discrimination between airway obstruction and centrally driven events. While they did it by using the nasal flow, we can derive it from the motion sensors on the chest. The advantage is that our sensors provide additional crucial data, as the amplitude of the signal and assessment of the effort required to provide the desired flow. Therefore, the motion sensors on the chest (instead of nasal airflow) may improve dramatically the sensitivity and specificity, especially during limited flow hypopnea (partial airway obstruction).

The integral of the magnitude of the amplitude of the chest wall movement and breath time length, named ATI, provided a better and more accurate insight on the amount of energy/effort exerted in the breathing process from the physiological point of view. While the TDi provides plain information on the relative change in the breathing effort, the ATI may correlate well with a non-invasive way for the quantification of the “work of breathing” and energy expenditure for breathing. The current ways to quantify work of breathing today in neonatal units involve invasive techniques that include the measurement of the transthoracic pressure (esophageal pressures) with a catheter and the use of a plethismograph or relative inductive plethismograpy (RIP) with chest and abdominal bands. These methods are mainly used for research purposes and for the clinical application most physicians will use only their clinical impression. The ATI can be obtained with small sensors suitable even for the smallest premature infants. Therefore, with the same sensors, a surrogate measure for the volume changes and for the EP can be obtained, the result of the inspiratory and expiratory loops can be followed and provide the means for the assessment of changes after a treatment or disease progression.

Apnea episodes are very frequent in the NICU, and the repeated occurrence of hypoxemic episodes frequently resulting from apnea, were linked to impaired neurodevelopmental outcome. The timely detection of apneic episodes from the events that preceded hypoxemia (as could be hypopnea), from their initiation, is of outmost importance. In the case of early detection and diagnosis, many of those events could be prevented, avoiding at least in part, a significant number of severe hypoxemic episodes and their consequences.

During CA, when no respiratory movements are present at all, current monitoring systems based on transthoracic impedance are able to provide an alarm. However, during OA or hypopnea, the episode will become evident only when bradycardia or hypoxemia are present, and the infants are already in severe distress or faint. Timely detection of apnea that evolves to hypoxemia and bradycardia in premature infants still remain in many cases unresolved as their incidence is still very high. We can speculate, that being able to timely detect the initiation of obstructive episodes and high or low resistance hypopnea, many of the hypoxemic episodes will be able to be avoided or ameliorated.

With prior art monitoring methods it is not possible to unambiguously detect obstructive apnea or distinguish between obstructive or central apnea without measuring airflow or nasal end-tidal CO₂ concomitantly to the respiratory effort. Nasal flow with the combination of thoracic bands, with or without abdominal bands, is currently in use for the assessment of apneic episodes in spite of the technical difficulties this imposes. However, absent flow in the airways one cannot differentiate between both types of apnea. Our results support the idea that distinction between Obstructive and Central apnea can be achieved using suggested technology amd method, since the combination and trends of the indices presented here: TDI, BTL and ATI, enable clear detection and classification of the apneic episodes.

Another reason for the lack of acceptability for routine use of chest and abdominal straps in the Neonatal ICU, are the conflicting results of the assessment of thoraco-abdominal synchrony. Synchrony was also tested using different ventilatory modalities in preterm infants with respiratory distress syndrome as summarized by Hammer et al (Hammer J, Newth CJ. Assessment of Thoraco-Abdominal Asynchrony. Paediatr Respir Rev 2009; 10:75-80). In their review, they summarized that this index lacked specificity for the diagnosis of OA and for the prediction of respiratory failure.

During the experiments we documented episodes of Near-Fatal Obstruction, where short obstructive apneic episodes evolved into an almost terminal event of central apnea. These episodes resemble the lack of arousal from hypoxemic events following obstruction observed in the clinics and suspected to be responsible, at least in part, for sudden death in susceptible subjects. Fatal or near-fatal obstruction may explain part of the events of ALTE or Sudden Death events suspected to occur following airway obstruction.

Events of OA are very frequent, and most of the time are uneventful, but in some cases, without the intervention of the caregivers, may become a cause for morbidity and mortality. In premature infants, pure obstructive events count for only around 10% of the apnea episodes. Another 40-50% is mixed apnea episodes, in which a CA precedes OA. Here we suggest that in some cases, following the obstructive event, in already compromised and borderline hypoxemic patients, fatal or near-fatal events of OA may be correlated with the subject's fatigue or with a defective arousal capability. As the number of episodes is high, and the subject's physiological baseline is depressed, the higher the chance for a fatal, terminal apneic event. The challenge of those types of events is for us to be able to provide an alarm immediately at the beginning of the obstructive episode, with enough time to react, in case that arousal does not occur and the subject cannot overcome the obstruction. The proposed indices in this study are able to provide this important information from the beginning of the obstructive episodes, allowing the team to provide the appropriate intervention before severe bradycardia and hypoxemia appear.

Limitations of the Study:

1—Our study was performed in a small animal model (adult rats) weighting 250-300 grams. They also had normal lungs and no inherent lung disease or problems with the control of breathing.

In conclusion, the real time continuous monitoring of the chest wall mechanics with a non-invasive device and miniature sensors can immediately detect all types of apneic episodes, and can easy classify the apneic episodes, in good correlation with the EP. Using the combination of these novel, but easy to understand mechanical indices, a surrogate for the Esophageal Pressure for the assessment and quantification of the respiratory effort can be considered as a good candidate to be incorporated to the NICU/PICU monitoring systems. The objective assessment of the trends in the work of breathing will also provide important information concerning the needs of an individual patient as well as the result of an intervention as could be certain respiratory support modality. Earlier detection and characterization of apnea-hypopnea events, may allow even a modest reduction in the enormous amount of hpoxemic events in the premature infant, accounting for a significant reduction in long term morbidity and mortality in this vulnerable population.

### Experiment 2

### Experimental Setup

Experiments were performed on six healthy New Zealand white rabbits with the approval of the Institutional Ethics Committee for the Care and Use of Animals. The rabbit was placed in the supine position and a tracheostomy was performed for use with a pressure-controlled ventilator (SLE 2000, SLE, Surrey, UK) under continuous positive airway pressure (CPAP). Three miniature accelerometers were attached to the rabbit as previously described (21). The acceleration signals were acquired using the Pneumonitor® (Pneumedicare, Yokneam, Israel).

Once arterial blood gasses were stabilized and the ventilation parameters were set, at least 5 minutes of baseline (BL) measurements were recorded. Four types of events were generated: fully obstructive apnea, partially obstructive apnea, hypoxia, and central apnea type event. Central type apnea (central apnea) was induced by succinylcholine as described previously (in the above rat experiments). Continuous mandatory ventilation was used to resuscitate the animal after cessation of breathing for 30 seconds. Three of the experiments began with obstructive apnea followed by hypoxia and three began with hypoxia followed by obstructive apnea. All were followed by central apnea.

Fully obstructive apnea was created by completely clamping the endotracheal tube. The full obstruction was maintained for a maximum of 30 seconds unless SpO₂<70%, HR<80, or MABP<40 occurred first. The maximal esophageal pressure (EP) during a full obstruction was used as a reference for the maximal effort exerted by each animal Two levels of partial obstructions, 50% and 25%, were used. A clamp was slowly tightened around the endotracheal tube until the EP rose to 50% or 25% of the maximal EP exhibited during full obstruction. Partial obstructions were maintained for 4 minutes. Blood gases were taken after 3 minutes of partial obstruction, when all the indices reached were stable.

Introducing nitrogen into the air mixture of the ventilator caused hypoxic events. The amount of nitrogen was increased until the desired fraction of oxygen (FiO₂) was achieved. The effects of three FiO₂ levels were investigated: 16%, 14% and 12%. The hypoxic event was maintained for 4 minutes. Blood gases were taken 3 minutes after the desired percentage of oxygen had been reached or at the end of events that ended prematurely due to animal distress.

### Signal Processing

The accelerometer signals were down-sampled to 250 Hz and low-pass filtered at 10 Hz, followed by feature extraction from a moving 10 s window with 1 s overlap. The following features were calculated and monitored to observe temporal: variance, entropy, kurtosis, phase difference, and respiratory rate.

K-means clustering was implemented in two stages to separate event types into baseline, 25% obstruction, and 16% hypoxia. Principal component analysis was performed and the first two principal components were chosen, representing at least 80% of the variance in the data. Additional detail on the calculation of the features implemented and specifics on how clustering was performed is provided in the online data supplement.

Signal processing, data analysis, and statistical analysis were performed using a commercial software package Matlab 2011 (The MathWorks Inc., Natick, Mass., USA). Results are presented as mean±STD. All parameters were assessed by a paired Wilcoxon sign-rank test and determined to be significant during an event when p<0.05.

### Results

The rabbits (n=6) weighed 3.79±0.18 Kg and were ventilated using CPAP with a continuous distending pressure of 4 cmH₂O.

The blood pressure (BP), oxygen saturation (SpO₂), determined by the pulse oximetry, end-tidal CO₂ (EtCO₂), esophageal pressure, flow rate, and respiratory rate, from impedance measurement, are presented from one experiment in Error! Reference source not found. The experiment was comprised of eight distinct events: hypoxia 16%, hypoxia 14%, hypoxia 12%, full obstruction 1, full obstruction 2, partial obstruction 50%, partial obstruction 25%, and central apnea. What is notable is that the mean arterial blood pressure remains relatively constant throughout all of the induced events. The SpO₂ decreases severely during hypoxia, as expected; however, during both partial obstructions, saturation remains almost indistinguishable from baseline despite the obvious increases in EtCO₂ and esophageal pressure while flow rate and respiratory rate decreased.

The statistical significance of the calculated parameters during a partial obstruction of 25% and hypoxia of 16% are shown in Table. The following figures show the parameters from one experiment and examples of accelerometer data of different event types. Error! Reference source not found. demonstrates changes in variance and entropy from the chest signals throughout the experiment. Both variance and entropy have a similar morphological response to each event type. Obstructive apnea and hypoxia both cause an increase in variance and entropy, while central apnea results in a decrease in both of these parameters.

In Error! Reference source not found, the kurtosis, chest-abdominal phase difference, and the respiratory rate, calculated from the accelerometer, are seen throughout the experiment. Kurtosis increases during obstructive events while decreases or remains unchanged during hypoxia. During central apnea, a decrease in kurtosis is seen on the abdominal sensor. The chest-abdominal phase difference increases during partial obstruction and decreases during the first two hypoxic events. During the most severe hypoxic event, phase difference was biphasic, initially decreased followed by an increase. Respiratory rate increased during hypoxic events and decreased during full obstruction, partial obstruction, and central apnea.

### Discussion

Monitoring the dynamics of the chest and abdomen by utilizing accelerometers has shown to provide unique features that are sensitive to small changes and specific enough to be able to differentiate between different types of respiratory events. The ability to distinguish between a partial obstruction and a hypoxic event is crucial in limiting the amount of false positives when monitoring infants for airway obstructions. Similar to an obstruction, a hypoxic event will cause respiratory distress, result in a breathing waveform with larger amplitude. RIP has been known to quantify TA asynchrony to assess the breathing effort from respiratory distress however, past techniques largely depended on the morphology of the breath. This dependency may lead to inaccurate measurements and may not be specific to differentiate between different types of respiratory distress.

Despite the variety and severity of the events induced throughout an experiment, Error! Reference source not found. shows a mean blood pressure that had minimal fluctuation. Observing SpO₂ during either partial obstruction would not have indicated any type of respiratory event although the animal was exerting up 50% of its maximal effort to overcome the obstruction.

A comparison was done between the weakest obstructive and hypoxic event that was induced. By implementing a two-stage classification technique it was possible to effectively identify and then classify between an obstruction and hypoxia. Abdominal kurtosis and chest-abdominal phase difference were the most crucial parameters in classifying an obstruction. Both parameters were only statistically significant during obstruction, shown in Table. By comparing the weakest of the events, it is expected that more severe partial obstructions or hypoxic events will be easier to detect and classify.

### Stage 1—Identification

Variance and entropy demonstrated large yet nonspecific changes from baseline and thus were utilized in the first stage to identify that a respiratory event was occurring. The mean changes of both even types were within one standard deviation of each other, suggesting that variance and entropy have low specificity and would be poor for separating between obstruction and hypoxia. The example in Error! Reference source not found. displays how both parameters respond similarly to an obstruction, hypoxia and central apnea. The severity of an event was also correlated well with each parameter. A lower FiO₂ or larger obstruction resulted in a greater response in both variance and entropy. When combined, variance and entropy were able to correctly detect all the deviations from baseline, whether obstructive or hypoxic.

The variance is a second order measurement and can be interpreted as the energy of the measured signal. This higher order statistic makes it highly sensitive to even small perturbations, which were observed with average increase of 150% and 400% in hypoxia and obstruction, respectively. Entropy is a statistical measurement that demonstrates the amount of information that is contained in the signal. When there is a large amount of information, the larger the unpredictability, resulting in higher entropy. During an obstruction or hypoxic event, the respiration and dynamics adapt to provide the required gas exchange. The entropy attempts to capture the additional information that is presented in the adaptation of the TA dynamics. The logarithmic calculation results in smaller changes nevertheless, about a 20% increase in entropy was seen in both obstruction in hypoxia.

### Stage 2—Classification

Once a significant change from baseline has been detected, the objective is to adeptly classify the type of event that has occurred. On average, kurtosis and the chest-abdominal phase difference during hypoxia remained unchanged from baseline. On the other hand, both parameters exhibited a substantial increase during partial obstruction. Error! Reference source not found. displays the distinct selectivity of both parameters to a partial obstruction. The respiratory rate is not inherently a novel parameter however, because it was calculated from the acceleration measuring the TA dynamics, it was has been included. As expected, the respiratory rate increased during hypoxia and decreased during an obstruction. The magnitude of respiratory rate change correlated well with the severity of the event induced.

If a PDF were to be constructed based on a measured signal, the shape of that distribution and how its peaks relate to the whole distribution would be captured by the kurtosis. Therefore, the kurtosis is capable of describing breathing waveform characteristics and how they change as a result of different modes of respiration.

### Signal Processing

The data acquisition system sampled the clinical channels at 5 kHz and the acceleration channels at 1 kHz. All of the data was downsampled to 250 Hz. The accelerometer signals were filtered with a zero phase 10 Hz low pass FIR filter and features were extracted from a moving 10 s window with 1 s overlap. In the following equations the symbol a, refers to a windowed and filtered acceleration signal.

The variance and the entropy of a signal were calculated to measure changes in the intensity of the signal as a result of different events. The variance of a signal was calculated to measure the intensity of the signal as a result of different events. The variance is:

var(a)=E[(a−μ)²]

where E[ ] is the expectation operator and μ is the mean of the signal. The entropy is a measure of randomness in the signal and was calculated by estimating the histogram of the windowed signal. The entropy, H, is then defined as:

H=Σp(a)log₂p(a)

where p is the probability of each value in the estimated histogram.

Kurtosis was implemented as a way to describe the shape of the windowed signal's probability distribution and how its tails relate to the peaks. Kurtosis was calculated using the standard definition of the fourth moment:

$$kurt ( a ) = \frac{E [ {( a - μ )}^{4} ]}{{( E [ {( a - μ )}^{2} ] )}^{2}}$$

The phase difference and the respiratory rate and were both calculated through the use of the analytic signal via the Hilbert transform. The chest and abdominal signals were treated as sinusoidal signals with the same frequency, but different phases. The phase difference was estimated from the combination of the Hilbert transform and the tangent identity from the difference of two angles described in Al-Angari H M, Sahakian A V. Automated Recognition of Obstructive Sleep Apnea Syndrome Using Support Vector Machine Classifier. Information Technology in Biomedicine, IEEE Transactions on 2012; 16:463-468, and Yang H, Tu Y, Zhang H, Yang K. A Hilbert Transform based method for dynamic phase difference measurement. 2012; 4141-4144. From the same Hilbert transform, several respiratory rates were calculated from the chest and abdominal sensors and then averaged. This respiratory rate was then compared to the impedance measurement and/or flow measurement to ensure accuracy.

Every event was referenced to the event's baseline value. The parameters of phase difference and respiratory rate were referenced as an absolute change in value from baseline, while variance, entropy, and kurtosis were referenced as a percentage change from baseline. For each event, one minute of baseline and one minute of an event was averaged to obtain 2 data points. This resulted in 6 baseline data points (1 for each animal) and 6 event data points for each event, per feature.

K-means clustering using a cosine distance measure was implemented in two stages to separate the data into baseline, 25% obstruction, and 16% hypoxia. Each feature was statistically normalized by its mean and standard deviation to remove any weights that occur from measuring different quantities. To reduce the dimensionality of the space, principal component analysis was performed and the first two principal components were chosen, representing at least 80% of the variance in the data.

The variance and entropy features were used for clustering in the first stage. Separation was performed on 24 observations (12 baseline, 6 obstruction, and 6 hypoxia) into two clusters: baseline and change in respiratory effort. For the second stage of clustering kurtosis, chest-abdominal phase, and respiratory rate was used to separate the observations previously classified as ‘change in respiratory effort’ into two clusters (obstruction and hypoxia).

TABLE 1
Summary of the stability and utility of the indices
used for the classification and quantification of induced
obstructive or central hypopnea/apnea events.

| | | | Central |
|---|---|---|---|
| | Base- | Total | Hypopnea/ |
| | line | Obstruction | Apnea |
| Tidal displacement index (TDi) | Stable | ↑ | ↓ |
| Breath Time Length (BTL) | Stable | ↑↑ | ↑ [symbol] |
| Amplitude Time Integral (ATI) | Stable | ↑↑↑ | ↓ |

TABLE 2
P-Values of Parameters

| Parameters | Obst. 25% | Hypoxia 16% |
|---|---|---|
| Chest Rad Variance | 0.0313 | 0.0313 |
| Chest Long Variance | 0.0313 | 0.0313 |
| Chest Rad Entropy | 0.0313 | 0.0313 |
| Chest Long Entropy | 0.0313 | 0.0313 |
| Chest-Abd Long Phase | 0.0313 | 0.5625 |
| Respiratory Rate | 0.0313 | 0.0313 |
| Chest Rad Kurtosis | 0.0313 | 0.4375 |
| Abd Kurtosis | 0.0313 | 0.1563 |

What is claimed is:

1. A device for monitoring and detecting apnea comprising:
a local acceleration chest sensor mountable on a chest of a patient for sensing local accelerations, called sensed local accelerations;
data amplification and filtration components;
a data acquisition system and data storage for data analysis and comparison with past data; and
a processor for calculating a respiratory effort index based on said sensed local accelerations and classifying a source of apnea as either central or obstructive by comparing the respiratory effort index to a baseline value defined when no apnea is present, wherein said respiratory effort index increases above the baseline for obstructive apnea and decreases below the baseline for central apnea.

2. The device according to claim 1, wherein said respiratory effort index comprises a Tidal Displacement index (TDi), comprising an amplitude of a tidal local displacement during a breath cycle at a measurement site of said local acceleration chest sensor.

3. The device according to claim 1, wherein said respiratory effort index comprises an Amplitude Time Integral (ATI), which comprises an integral of an instantaneous tidal displacement over time for an entire respiratory cycle.

4. The device according to claim 1, comprising two local acceleration chest sensors for placement on left and right sides of the chest and a local acceleration abdomen sensor for placement near a diaphragm of the patient, wherein said processor determines synchrony of breathing movements between both sides of the chest and the abdomen, wherein during obstruction apnea, motion of the chest and the diaphragm are slightly out of phase or in complete opposite phase.

5. A method for monitoring and detecting apnea comprising:
sensing local accelerations, called sensed local accelerations with a local acceleration chest sensor mounted on a chest of a patient; and
calculating a respiratory effort index based on said sensed local accelerations and classifying a source of apnea as either central or obstructive by comparing the respiratory effort index to a baseline value defined when no apnea is present, wherein said respiratory effort index increases above the baseline for obstructive apnea and decreases below the baseline for central apnea.

6. The method according to claim 5, wherein said respiratory effort index comprises a Tidal Displacement index (TDi), comprising an amplitude of a tidal local displacement during a breath cycle at a measurement site of said local acceleration chest sensor.

7. The method according to claim 5, wherein said respiratory effort index comprises an Amplitude Time Integral (ATI), which comprises an integral of an instantaneous tidal displacement over time for an entire respiratory cycle.

8. The method according to claim 5, comprising sensing local acceleration on left and right sides of the chest and a diaphragm of the patient, wherein during obstruction apnea, motion of the chest and the diaphragm are slightly out of phase or in complete opposite phase.
